(19) United States
(12) Patent Application Publication
(10) Pub. No.: US 2025/0241853 A1
(43) Pub. Date: Jul. 31, 2025

(54) PREPARATION METHOD AND APPLICATION FOR OIL-INWATER NANOEMULSION MATERIAL

(71) Applicant: Guizhou Medical University, Anshun (CN)

(72) Inventors: Zuquan HU, Anshun (CN); Liang HONG, Anshun (CN); Jianman WANG, Anshun (CN); Meng LUO, Anshun (CN); Yuxue YAN, Anshun (CN); Yali LUO, Anshun (CN); Jiangmin LI, Anshun (CN)

(73) Assignee: Guizhou Medical University, Anshun (CN)

(21) Appl. No.: 18/806,731

(22) Filed: Aug. 16, 2024

(30) Foreign Application Priority Data
Jan. 31, 2024 (CN) 202410134061.0

(51) Int. Cl. A61K 9/107; A61K 31/12; A61K 31/352; A61P 35/00

(52) U.S. Cl. CPC A61K 9/1075; A61K 31/12; A61K 31/352; A61P 35/00

(57) ABSTRACT

A preparation method and an application for an oil-in-water nanoemulsion material are provided, the method is a new method combining sonication-phase inversion composition-phase inversion temperature, S-PIC-T method, i.e., the combined application of the sonication, the phase inversion composition method and the phase inversion temperature develops a new type nano-preparation with small droplet size, high loading hydrophobic small molecule substances, and stable physical and chemical properties. Therefore, in the present disclosure, the sonication in the high-energy method is combined with PIC and PIT in the low-energy method, because the sonication does not produce high heat, the amount of surfactant is reduced, and the size of emulsion droplets is reduced while the energy consumption is low, while PIC and PIT reduce production costs and protect the biological activity of hydrophobic bioactive substances.

### CROSS REFERENCE TO THE RELATED APPLICATIONS

This application is based upon and claims priority to Chinese Patent Application No. 202410134061.0, filed on Jan. 31, 2024, the entire contents of which are incorporated herein by reference.

### TECHNICAL FIELD

The present disclosure relates to a preparation method and an application for an oil-in-water nanoemulsion material, which belongs to the technical field of nanoemulsion material.

### BACKGROUND

Nanoemulsion (NE) refers to a thermodynamic stable and isotropic dispersion system with a transparent or translucent appearance and particle size of 1-100 nm formed by two immiscible solutions under the action of the surfactant or cosurfactant. According to its structure, the nanoemulsion can be divided into water in oil type (W/O), oil in water type (O/W) and bicontinuous phase type (W/O/W or O/W/O). Its structural type depends on the proportion and properties of each phase, and under certain conditions, they can be converted into each other.

One of the important applications of nanoemulsion is the preparation of polymer nanoparticles taking monomers as dispersed phases (so-called nanoemulsion polymerization). Nanoemulsion droplets can be seen as small nanoreactors. In addition to being used as a reaction medium, the nanoemulsion is widely used in chemistry, pharmacy, cosmetics and other fields. The nanoemulsion can also be used as drug delivery systems for multiple routes of administration. External intestinal (i.e. injection) administration of the nanoemulsion is used for a variety of purposes, such as the preparation of low-energy nanoemulsion and their research as carriers for phenylethylresorcinol and astaxanthin, including nutrition (such as the application of fats, carbohydrates, vitamins, etc.), controlling drug release and targeted delivery of drugs to specific parts of the body, delivering vaccines, and serving as gene carriers. Due to the strict requirements for this intravenous route of administration, especially the requirement that the droplet size of the preparation must be less than 1 m, the nanoemulsion has advantages for intravenous administration. The benefits of the nanoemulsion in the oral administration of drugs have also been reported, and the absorption of the emulsion in the gastrointestinal tract is related to its droplet size. Cationic nanoemulsion is used as DNA vaccine carriers through the lung route for drug administration. They are also candidates for delivering drugs through the skin or DNA plasmids. The aesthetic characteristics of the nanoemulsion, including low viscosity, transparent appearance, droplet size less than 100 nm, and high physical stability (ability to resist stratification and settlement), make the application of the nanoemulsion in cosmetics particularly attractive. Because it can increase penetration and skin hydration, the nanoemulsion can add great value to skin care products.

In the traditional preparation methods of the nanoemulsion, high-energy or low-energy methods are usually used for preparation. The high-energy method uses specialized mechanical equipment (such as sonar, ultrasound, microfluidics, and high-pressure valve homogenizers), to form small droplets with nanometer size, which is suitable for the preparation of the nanoemulsion using a small amount of surfactant and oil. However, the use of large equipment in the high-energy method can cause problems such as high costs and significant damage to bioactive chemicals. The low-energy method mainly relies on the combination of surfactant-oil-water to spontaneously form small droplets with nanometer size under appropriate environment or conditions. The low-energy method usually includes a phase inversion composition method (PIC), a phase inversion temperature method (PIT) and spontaneous emulsification, which has the advantages of lower cost and less damage to bioactive chemicals. However, the types of surfactant and oils that can be utilized in the preparation of the nanoemulsion by the low-energy method are limited, it is usually necessary to use a large number of surfactant to form the nanoemulsion with nanometer size, which is inefficient, at the same time, the presence of a large number of surfactant may have serious biosafety problems.

### SUMMARY

In order to solve the above technical problems, the present disclosure provides a preparation method and an application for an oil-in-water nanoemulsion material, and the preparation method has the advantages of simple operation, low energy consumption, small damage to the activity of hydrophobic small molecular substances, and can effectively promote the emulsification effect of oil phase and water phase, effectively improve the Ostwald ripening phenomenon in solution, uniformly disperse solid particles, improve the stability of emulsion, and realize the application of hydrophobic bioactive substances in biomedicine.

The present disclosure is constructed as follows: a preparation method for an oil-in-water nanoemulsion material, including the following steps:
Step 1. first, adding a certain amount of hydrophobic small molecule substances, oil and surfactant to a transparent glass bottle, and then fixing the transparent glass bottle in an ultrasonic cleaner and performing a water bath ultrasonic treatment for 20 min;
Step 2. placing the transparent glass bottle after the water bath ultrasonic treatment on a magnetic stirrer and stirring for 20 min, then, under the condition of continuous stirring, adding aqueous phase (phosphate buffered saline (PBS)) dropwise into the bottle using a peristaltic pump, and continuing stirring for 30 minutes after completing the addition of aqueous phase (PBS);
Step 3. putting a certain amount of water into a beaker, and then placing the beaker on a magnetic stirrer with a heating device, heating the water in the beaker to 60° C. in advance and maintaining a constant temperature, at this time, placing the transparent glass bottle in the beaker with water, and continuously stirring the emulsion in the transparent glass bottle for 20 min at 60° C.;
Step 4. quickly placing the transparent glass bottle processed by the above operation in an ice water bath to perform cooling, after lowering a temperature to 5° C., preparing and obtaining the oil-in-water nanoemulsion loaded with hydrophobic small molecules, and finally, storing the oil-in-water nanoemulsion in a 4° C. refrigerator.

In the above method, calculated by mass percentage, proportions of hydrophobic small molecule substances, oil, surfactant, and aqueous phase (PBS) are 1%, 6%, 4%, and 89%, respectively.

In the above method, the hydrophobic small molecule substances are isoliquiritigenin, curcumin, myricetin, apigenin or naringenin.

In the above method, in step 1, placing in a central position of a liquid level in the ultrasonic cleaner after wrapping the transparent glass bottle in tin paper, and making a bottle body of the transparent glass bottle enter 1.5 cm below the liquid level.

In the above method, a pH of the aqueous phase (PBS) is 7.4.

In the above method, a power of the ultrasonic cleaner is 40 kHz, a speed of the magnetic stirrer is 750 rpm, and a speed of the peristaltic pump is 22 rpm.

At the same time, the present disclosure further provides an oil-in-water nanoemulsion material obtained by the preparation method of the oil-in-water nanoemulsion material.

In addition, the present disclosure further provides an application for the above oil-in-water nanoemulsion material in preparing drugs for treating breast cancer.

Due to the adoption of the above technical solutions, the present disclosure has the following advantages: the present disclosure is a new method combining sonication-phase inversion composition-phase inversion temperature, S-PIC-T method, i.e., the combined application of the sonication, the phase inversion composition method and the phase inversion temperature develops a new type nano-preparation with small droplet size, high loading hydrophobic small molecule substances, and stable physical and chemical properties. Therefore, in the present disclosure, the sonication in the high-energy method is combined with PIC and PIT in the low-energy method, because the sonication does not produce high heat, the amount of surfactant is reduced, and the size of emulsion droplets is reduced while the energy consumption is low, while PIC and PIT reduce production costs and protect the biological activity of hydrophobic bioactive substances, which not only improves the preparation efficiency of oil-in-water nanoemulsion carriers, shortens the preparation time, but also further enhances its application in the fields of chemistry, pharmacy, and cosmetics under the premise of protecting the biological activity of hydrophobic substances.

### BRIEF DESCRIPTION OF THE DRAWINGS

FIG. 1A is a loading rate effect diagram of the oil-in-water nanoemulsion of the present disclosure;

FIG. 1B is a loading rate effect diagram of isoliquiritigenin microemulsion;

FIG. 1C is a loading rate effect diagram of iRGD-modified isoliquiritigenin hybrid nanoparticles;

FIG. 1D is a loading rate effect diagram of TPGS-modified isoliquiritigenin liposomes;

FIG. 2 shows an electron microscopy image of the oil-in-water nanoemulsion loaded with isoliquiritigenin of the present disclosure;

FIG. 3 shows a particle size distribution diagram of the oil-in-water nanoemulsion loaded with isoliquiritigenin of the disclosure measured by nanoparticle size analyzer;

FIG. 4 is an effect diagram of the oil-in-water nanoemulsion of the present disclosure in anti-breast cancer experiments in vivo and in vitro;

FIG. 5 shows an electron microscope image of the oil-in-water nanoemulsion of the present disclosure after co-culture with mouse breast cancer 4T1 cells;

FIG. 6 shows a tumor growth curve diagram in mice in vivo after treatment with the oil-in-water nanoemulsion of the present disclosure; and

FIG. 7 shows an appearance diagram of tumors extracted from mice after treatment with the oil-in-water nanoemulsion of the present disclosure.

### DETAILED DESCRIPTION OF THE EMBODIMENTS

In order to make the purpose, technical solutions and advantages of the invention clearer, the following is a further detailed description of the present disclosure in combination with the accompanying drawings and embodiments.

### EMBODIMENT

This embodiment took the hydrophobic small molecule substance-isoliquiritigenin as an example, and the embodiment of the present disclosure was as follows:

A preparation method for an oil-in-water nanoemulsion material, including the following steps:
Step 1. first, calculate by mass percentage, 1% isoliquiritigenin, 6% oil and 4% surfactant was added into a 30 mL transparent glass bottle, then the transparent glass bottle was wrapped in tin paper and placed in a central position of a liquid level in an ultrasonic cleaner, and a bottle body of the transparent glass bottle was made to enter into the liquid level under 1.5 cm, then a water bath ultrasonic treatment was performed, a power of the ultrasonic cleaning machine was 40 kHz, and a water bath ultrasonic treatment time was 20 min;
Step 2. the transparent glass bottle after the water bath ultrasonic treatment was placed on a magnetic stirrer and stirred for 20 min, a rotation speed of the magnetic stirrer was 750 rpm, after stirring for 20 minutes, under continuous stirring conditions, 10.8 mL of aqueous phase (PBS) was added dropwise to a center of the transparent glass bottle using a peristaltic pump, and a pH of the aqueous phase (PBS) was 7.4, this process promoted the gradual transition from the water-in-oil nanoemulsion (W/O) to the oil-in-water nanoemulsion (O/W), and a rotation speed of the peristaltic pump is 22 rpm; and after the dropwise addition of the aqueous phase (PBS) was complete, the stirring was continued for 30 min;
Step 3. a beaker was filled with 150 mL of water, and then the beaker was placed on a magnetic stirrer with a heating device, the water in the beaker was heated to 60° C. in advance and maintained a constant temperature, at this time, the transparent glass bottle was placed in the beaker filled with water, and the emulsion in the transparent glass bottle was continuously stirred for 20 min at 60° C., and a rotation speed of the magnetic stirrer was 750 rpm; and
Step 4. the transparent glass bottle after the above operation was quickly placed in an ice water bath to perform cooling, when the temperature was reduced to 5° C., the oil-in-water nanoemulsion loaded with isoliquiritigenin was prepared, and finally, the oil-in-water nanoemulsion was stored in a 4° C. refrigerator.

The preparation principle of the preparation method of the oil-in-water nanoemulsion material was as follows:

The combined application of high energy method and low energy method:

the high-energy method used ultrasonic waves to generate local microturbulence, making the dispersed phase unstable, the cavitation cavities in the continuous phase were generated during the rarefied period of the wave and continued to increase in the rarefaction period of the wave until collapsing in the compression period, resulting in high pressure and high shear, and the dispersed phase was broken into severe turbulence of nano-scale water droplets by a liquid jet, which tended to further break large-sized droplets into nano-sized small droplets.

In the low-energy method, the PIC method utilized changing the proportion of each component in the system to undergo phase transition. The nanoemulsion was formed from an unstable W/O system to a stable O/W system through aqueous phase dilution. The phase inversion rate might be adjusted according to the amount of water phase added, the speed of water phase added, the viscosity of oil, the concentration of surfactant and its hydrophilic-lipophilic equilibrium value. The PIT method was based on the temperature-induced change in the spontaneous curvature of the surfactant, which caused the surfactant to spontaneously move from the oil phase to the aqueous phase to produce the nanoemulsion. When the temperature was below PIT, the nonionic surfactant was mostly hydrophilic, forming a kind of milky white emulsion; as the temperature increased, the nonionic surfactant gradually became lipophilic and was dissolved by the oil phase; when the temperature was at the PIT, the interfacial curvature and interfacial tension decrease, which provided favorable conditions for the establishment of microemulsion in an equilibrium state, to form the bicontinuous microemulsion; and when the temperature was further increased above PIT, the system undergoes phase inversion, the aqueous phase was dispersed in the mixture of oil and surfactant (the surfactant was lipophilic at this temperature), to produce the nanoemulsion.

Through the combined application of the high-energy method and the low-energy method, the nanoemulsion with small droplet size, stable physical and chemical properties, and high hydrophobic small molecule loading were prepared.

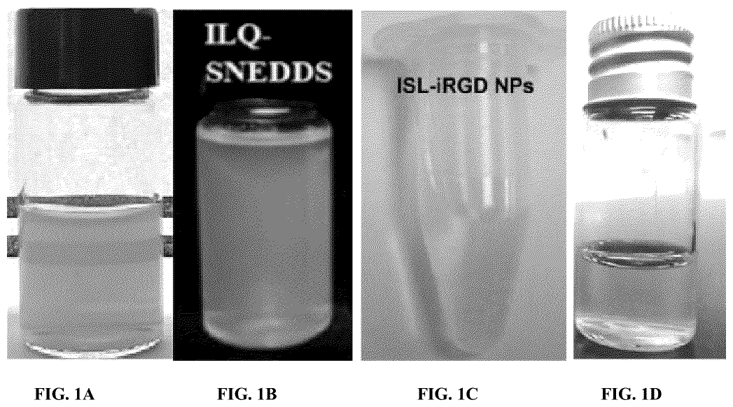

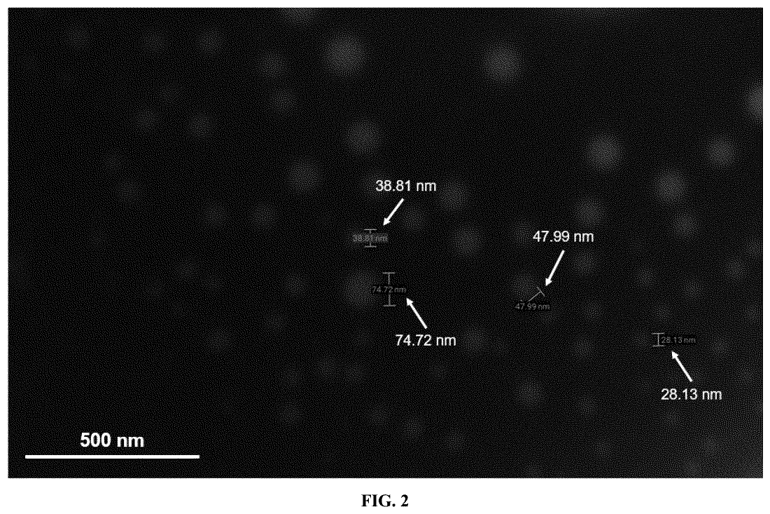

In order to verify the loading effect of the oil-in-water nanoemulsion A of the present disclosure, isoliquiritigenin microemulsion D1, iRGD-modified isoliquiritigenin hybrid nanoparticle D2 and TPGS-modified isoliquiritigenin liposome D3 were compared with the oil-in-water isoliquiritigenin nanoemulsion A of the present disclosure. Referring to FIGS. 1A-1D, the oil-in-water nanoemulsion A of the present disclosure had a uniform loading effect on isoliquiritigenin, while the loading effect of isoliquiritigenin microemulsion D1, iRGD-modified isoliquiritigenin hybrid nanoparticle D2 and TPGS-modified isoliquiritigenin liposome D3 on isoliquiritigenin had poor uniform; and meanwhile, referring to FIG. 2, the oil-in-water nanoemulsion A of the present disclosure exhibited a narrower small-size droplet distribution. Referring to Table 1, from Table 1, it might be seen the encapsulation rate and concentration of four types of nanoemulsion on isoliquiritigenin, wherein, the encapsulation rate and concentration of the oil-in-water nanoemulsion A of the present disclosure were the highest.

TABLE 1
Encapsulation rate and concentration of four
types of nanoemulsion on isoliquiritigenin

| Code | | Encapsulation | Isoliquiritigenin | |
|---|---|---|---|---|
| name | Name | rate | concentration | |
| A | Oil-in-water nanoemulsion | 100% | 10 | mg/mL |
| D1 | Isoliquiritigenin | 92.50% ± 0.45% | 0.864 | mg/mL |
| | microemulsion | | | |
| D2 | iRGD modified | 90.80% ± 1.50% | 0.120 | mg/mL |
| | isoliquiritigenin hybrid | | | |
| | nanoparticles | | | |
| D3 | TPGS modified | 97.33% ± 0.40% | 0.830 | mg/mL |
| | isoliquiritigenin liposomes | | | |

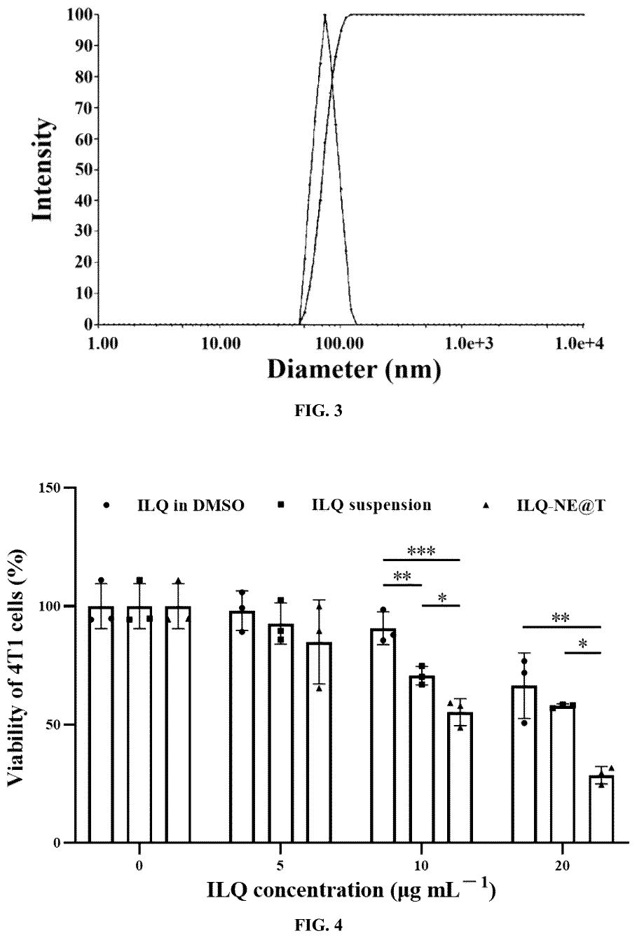

Referring to FIG. 3, the droplet size of the oil-in-water nanoemulsion was 66.72±1.31 nm; the polydispersity index is 0.098±0.016, the smaller the value was, the more uniform the size of the nanoemulsion droplets was, the more uniform the size of the nanoemulsion might avoid the attraction of large particles to small particles, that was, to avoid the Ostwald ripening phenomenon, which led to the further increase of large particles to form precipitation. Therefore, the oil-in-water nanoemulsion of the present disclosure might uniformly disperse solid particles and improve the stability of the emulsion.

In addition, the oil-in-water nanoemulsion of the present disclosure had an obvious effect in anti-breast cancer experiments in vitro and in vivo.

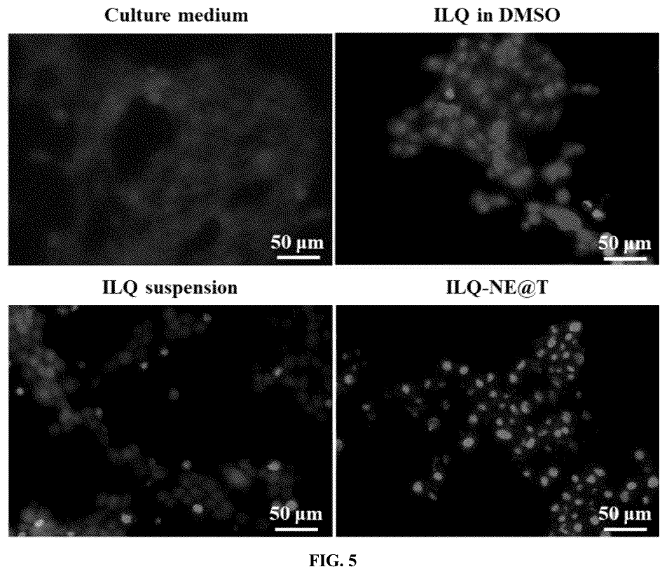

Referring to FIG. 4, FIG. 4 showed an effect of the oil-in-water nanoemulsion of the present disclosure after co-culture with mouse breast cancer 4T1 cells on the activity of 4T1 breast cancer cells. It might be seen from FIG. 4 that when the concentration of isoliquiritigenin was 10 g/mL and 20 g/mL, it had a stronger killing effect on cancer cells than the isoliquiritigenin dispersion that was not encapsulated by the nanoemulsion carrier; wherein ILQ-NE@T adopted the oil-in-water nanoemulsion loaded with isoliquiritigenin obtained by the preparation method of the present disclosure; and ILQ in DMSO and ILQ suspension represented two different isoliquiritigenin dispersions that were not encapsulated by nanoemulsion carriers.

Referring to FIG. 5, after the oil-in-water nanoemulsion of the present disclosure was co-cultured with mouse breast cancer 4 T1 cells, the death and survival of 4T1 breast cancer cells were observed by staining of live and dead cells. The stronger the red fluorescence signal was, the more dead cells were, and the stronger the green fluorescence signal was, the more living cells were. It might be seen from FIG. 5 that the 4T1 breast cancer cells treated with ILQ-NE@T basically died, and the isoliquiritigenin dispersion that was not encapsulated by the nanoemulsion carrier had little effect on the survival of 4T1 breast cancer cells; wherein Culture medium was a medium without isoliquiritigenin, and ILQ-NE@T adopted the oil-in-water nanoemulsion of the present disclosure; and ILQ in DMSO and ILQ suspension represented two different isoliquiritigenin dispersions that were not encapsulated by nanoemulsion carriers.

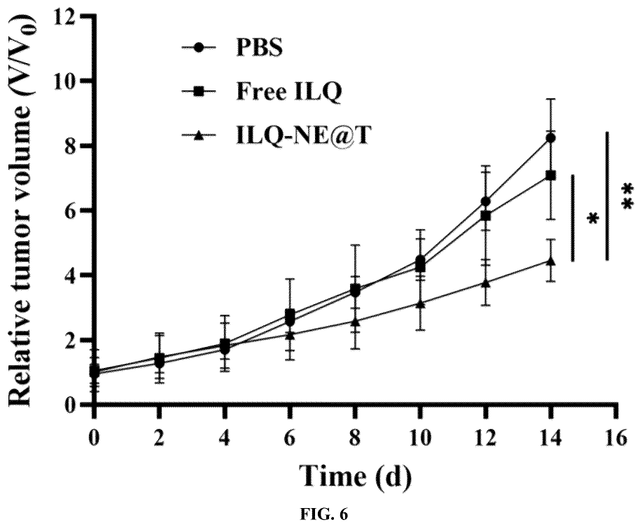

Referring to FIG. 6, FIG. 6 showed a tumor growth curve in mice treated with the oil-in-water nanoemulsion of the present disclosure during the 14-day treatment period. It might be seen from FIG. 6 that the tumor growth rate after treatment with ILQ-NE @T was significantly slower than that after treatment with PBS and isoliquiritigenin dispersion not encapsulated by nanoemulsion carrier; wherein PBS was phosphate buffered saline, and ILQ-NE@T adopted the oil-in-water nanoemulsion loaded with isoliquiritigenin obtained by the preparation method of the present disclosure; and Free ILQ represent isoliquiritigenin dispersion that was not encapsulated by nanoemulsion carrier.

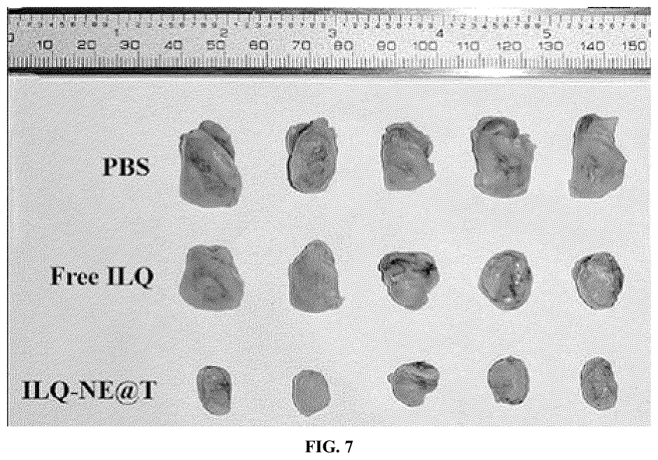

Referring to FIG. 7, FIG. 7 showed an appearance diagram of the tumor in mice after treatment with the oil-in-water nanoemulsion of the present disclosure. It might be seen from FIG. 6 that the tumor volume and size after treatment with ILQ-NE @T were significantly smaller than those after treatment with PBS and isoliquiritigenin dispersion not encapsulated by nanoemulsion carrier; wherein PBS was phosphate buffered saline, and ILQ-NE@T adopted the oil-in-water nanoemulsion loaded with isoliquiritigenin obtained by the preparation method of the present disclosure; and Free ILQ represented isoliquiritigenin dispersions that were not encapsulated by nanoemulsion carriers.

What is claimed is:

1. A preparation method for an oil-in-water nanoemulsion material, comprising the following steps:
step 1: first, adding a predetermined amount of hydrophobic small molecule substances, an oil, and a surfactant to a transparent glass bottle, and then fixing the transparent glass bottle in an ultrasonic cleaner and performing a water bath ultrasonic treatment for 20 min;
step 2: placing the transparent glass bottle after the water bath ultrasonic treatment on a magnetic stirrer and stirring for 20 min, then, under a condition of a continuous stirring, adding an aqueous phase dropwise into the transparent glass bottle using a peristaltic pump, and continuing stirring for 30 minutes after completing an addition of the aqueous phase, wherein the aqueous phase is phosphate buffered saline (PBS);
step 3: putting a predetermined amount of water into a beaker, and then placing the beaker on a magnetic stirrer with a heating device, heating the water in the beaker to 60° C. in advance and maintaining a constant temperature, at this time, placing the transparent glass bottle in the beaker with the water, and continuously stirring an emulsion in the transparent glass bottle for 20 min at 60° C.; and
step 4: quickly placing the transparent glass bottle processed by the step 3 in an ice water bath for a cooling, after lowering a temperature to 5° C., preparing and obtaining an oil-in-water nanoemulsion loaded with the hydrophobic small molecule substances, and finally, storing the oil-in-water nanoemulsion in a 4° C. refrigerator.

2. The preparation method for the oil-in-water nanoemulsion material according to claim 1, wherein calculated by a mass percentage, proportions of the hydrophobic small molecule substances, the oil, the surfactant, and the aqueous phase are 1%, 6%, 4%, and 89%, respectively.

3. The preparation method for the oil-in-water nanoemulsion material according to claim 2, wherein the hydrophobic small molecule substances are isoliquiritigenin, curcumin, myricetin, apigenin, or naringenin.

4. The preparation method for the oil-in-water nanoemulsion material according to claim 1, wherein in the step 1, the transparent glass bottle is placed in a central position of a liquid level in the ultrasonic cleaner after wrapping the transparent glass bottle in a tin paper, and a bottle body of the transparent glass bottle enters 1.5 cm below the liquid level.

5. The preparation method for the oil-in-water nanoemulsion material according to claim 1, wherein a pH of the aqueous phase is 7.4.

6. The preparation method for the oil-in-water nanoemulsion material according to claim 1, wherein a power of the ultrasonic cleaner is 40 kHz, a speed of the magnetic stirrer in the step 2 is 750 rpm, and a speed of the peristaltic pump is 22 rpm.

7. An oil-in-water nanoemulsion material obtained by the preparation method according to claim 1.

8. A method of preparing a drug for treating a breast cancer, comprising using the oil-in-water nanoemulsion material according to claim 7.

9. The oil-in-water nanoemulsion material according to claim 7, wherein in the preparation method, calculated by a mass percentage, proportions of the hydrophobic small molecule substances, the oil, the surfactant, and the aqueous phase are 1%, 6%, 4%, and 89%, respectively.

10. The oil-in-water nanoemulsion material according to claim 9, wherein in the preparation method, the hydrophobic small molecule substances are isoliquiritigenin, curcumin, myricetin, apigenin, or naringenin.

11. The oil-in-water nanoemulsion material according to claim 7, wherein in the step 1 of the preparation method, the transparent glass bottle is placed in a central position of a liquid level in the ultrasonic cleaner after wrapping the transparent glass bottle in a tin paper, and a bottle body of the transparent glass bottle enters 1.5 cm below the liquid level.

12. The oil-in-water nanoemulsion material according to claim 7, wherein in the preparation method, a pH of the aqueous phase is 7.4.

13. The oil-in-water nanoemulsion material according to claim 7, wherein in the preparation method, a power of the ultrasonic cleaner is 40 kHz, a speed of the magnetic stirrer in the step 2 is 750 rpm, and a speed of the peristaltic pump is 22 rpm.
